(19) United States
(12) Patent Application Publication
(10) Pub. No.: US 2024/0404050 A1
(43) Pub. Date: Dec. 5, 2024

(54) SYSTEM AND METHOD FOR REMOVAL OF CORNEAL SPECULAR REFLECTIONS IN EYE IMAGING

(71) Applicant: Johnson & Johnson Surgical Vision, Inc., Irvine, CA (US)

(72) Inventors: Li Chen, San Jose, CA (US); Jianming Xu, San Jose, CA (US); Richard Hofer, Santa Cruz, CA (US)

(21) Appl. No.: 18/679,349

(22) Filed: May 30, 2024

Related U.S. Application Data
(60) Provisional application No. 63/505,634, filed on Jun. 1, 2023.

(51) Int. Cl. G06T 7/00; A61B 90/00; A61B 90/30; A61F 9/008

(52) U.S. Cl. CPC G06T 7/0012; A61B 90/30; A61B 90/361; A61F 9/008; A61B 2090/309; G06T 2207/30041

(57) ABSTRACT

Methods and devices are disclosed for pulsed LED illumination, image capture processing to remove corneal specular reflections in torsion images for iris registration. A system may first and second infrared LED light sources and a torsion camera to capture images during illumination of a patient's eye based on a pulsed synchronization signal. Within a period of time, a first image is captured having a reflection from the first LED light source and then a second image is captured having a reflection from the second LED light source. The first and second images are processed to remove respective areas having corneal specular reflections and combined into a third image not including any specular reflections. Various embodiments are disclosed including implementations with excimer and femtosecond laser eye surgery platforms.

### CROSS-REFERENCE TO RELATED APPLICATIONS

This application claims the benefit under 35 U.S.C. § 119 (e) of U.S. Provisional Patent Application No. 63/505,634, filed Jun. 1, 2023, which is incorporated herein by reference in its entirety.

### FIELD OF INVENTION

Aspects of the invention generally relate to eye surgical systems and methods, and more particularly to, systems and methods for imaging and tracking of eye measurements during ophthalmic surgeries.

### BACKGROUND

Ophthalmic surgeries to perform vision correction have used laser-based systems in a variety of applications. For example, laser refractive surgeries such as phototherapeutic keratectomy (PRK) and laser-assisted in situ keratomileusis (LASIK) have been used to correct the refractive errors in patients' eyes. In certain applications, lasers may be driven by a treatment plan from the preoperative eye exam measured by a diagnostic device to achieve a desired change in corneal shape for vision correction. Wavefront measurement systems have been previously utilized to measure refractive characteristics of a particular patient's eye and an ablation pattern may be determined from the measurements to correct refractive errors.

For certain procedures, the eye may undergo cyclotorsional eye movements between eye examination/measurement and laser surgery, whereby portions of the eye may change or shift. For example, the pupil center may shift with respect to the surface of the cornea as pupil size changes from scotopic to photopic lighting conditions. Iris registration (IR) has been used to compensate for the pupil center shift and cyclorotation of the eye between a wavefront examination and subsequent laser surgery. In some systems, a torsion camera is used to capture images for IR. However, these systems may incur undesirable corneal specular reflections during imaging as a result of, for example, light sources used to illuminate the eye for imaging purposes. Specular reflections from natural or artificial sources may induce high-light intensity reflections and/or reduce image detail which may interfere with position detection and motion compensation. For example, specular reflections in eye image may reduce an area that may be suitably imaged in iris areas having one or more light reflections. It is desirable to have a maximum iris area available for IR to compensate eye movements between eye exam and laser treatment.

One example approach to address undesirable specular light reflections in eye surgeries is disclosed in U.S. Published Application 2009/0275929A1, whereby a light source and/or a camera lens include polarization filters to polarize the light from the source and/or to the imaging device to prevent reflections from being imaged. This approach increases mechanical complexity due to linear polarizers installation as well as increased expense in adding/modification of hardware. Furthermore, the polarized illumination approach has disadvantages including darker imaging from polarized light waves and consequently may require a higher light density be used during imaging. An increased light density requires increased current to drive the light source, thereby resulting in greater power consumption and increased heating of the light source such as an infrared LED, thereby reducing lifespan of the light source. A more effective and cost-sensitive method of removing corneal specular reflections for eye imaging remains desirable.

### SUMMARY

Aspects of the disclosed embodiments may generally relate to methods and systems for improving eye tracking and motion compensation during an ophthalmic laser procedure. According to one aspect, a method of imaging for an eye for a surgical procedure may include capturing a first image of the eye while the eye is illuminated with a first light source and, within a subsequent time period from the capturing of the first image, capturing a second image of the eye while the eye is illuminated with a second different light source. The first and second images may be processed into a third image to remove reflections associated with the first light source and the second light source to achieve an image without corneal specular reflections.

According to some aspects, the first light source is an auxiliary infrared light emitting diode (LED) and the second light source is an ocular dexter (OD) or oculus sinister (OS) infrared LED for respective right eye or left eye imaging. The first and second light sources are displaced in separate locations of a laser diagnostic and/or treatment light source, so as to cause a displacement between a specular reflection in the first image and a specular reflection in the second image. In one example, an imaged area of the eye not having a reflection in the second image may be superimposed or utilized for iris tracking in an area of the first image that does include a reflection, and vice versa, to produce the third image where a full imaged area of the eye absent any specular reflections may be presented, used or implemented for tracking and or measurement and subsequent treatment.

According to one aspect, a timed pulse of a specified frequency may be used to trigger illumination and de-illumination of the first light source and an offset of the rising edge of the timed pulse is used to trigger capturing the first image. A subsequent second timed pulse of the specified frequency may similarly trigger illumination and de-illumination of the second light source, and an offset from the rising edge of the second timed pulse triggers capturing of the second image. In certain, non-limiting embodiments, a frequency cycle for the first and second time pulses is set above 60 Hz, for example, at approximately for 100 Hz to enable a frame per second capturing of first and second images to occur with minimized potential eye movement between first and second captured images. According to various aspects, the third image is used to track one or more iris locations for wavefront measurement and subsequent laser refractive treatment surgery, such as a LASIK procedure.

According to further aspects, a surgical system for imaging an area of eye of a patient is disclosed. The surgical system may include an imaging light configured to intermittently trigger illumination of a first light source for a first time period and illumination of a second light source for a second time period, different than the first time period. The system may further include a camera, such as a torsion camera, to produce a first image of the area during the first time period and a second image of the area during the second time period. The system may further include a processor configured to generate a third image based on the first image and second image, including removal of one or more light reflections from the area from the first image based on the same area of the second image not including light reflections of the first image. The processor further generates the third image by removing differently located light reflections in areas of the second image with the same areas of the first image not including the differently located light reflections. The third image is generated without any corneal specular reflections in this manner. The system may include additional components for processing, measurement and treatment as detailed further below, but generally, may operate based on a timing circuit or logic to initiate pulses at a specific frequency to intermittently drive illumination and de-illumination of the first and second light source and capturing of the first and second images during respective illuminations by the first and second light sources.

According to some aspects, the area of an eye for imaging is an iris, although the embodiments are not so limited. In one example system, the first light source and the second light sources of the imaging light are separated and arranged in the imaging light to produce corneal specular reflections at different locations of the eye area, such as an iris, during first and second image capture. The first and second time period may occur in a cycle of greater than 60 Hz to avoid imaging inconsistencies such as eye movements between capturing of first and second images.

In some embodiments, the system imaging light may further include a third light source, e.g., OD LED or OS LED, along with auxiliary light source, to capture respective first and second images of an opposing eye of the patient and generate a further image of the opposing eye absent corneal specular reflections. According to some aspects, the first light source and the second light source are light emitting diodes (LEDs), and in some embodiments, infrared LEDs, although the embodiments are not so limited as different types of light sources or bulbs may also be used. Additionally, the surgical system may be for laser wavefront measurement and laser refractive surgery such as an excimer laser system for LASIK procedure or component thereof, although the embodiments are not limited in this respect and may be used in any medical diagnostic or treatment system where similar advantages may suitably be derived.

### BRIEF DESCRIPTION OF THE DRAWINGS

Aspects, features and advantages of the disclosed embodiments will become more apparent from the subsequent detailed description in reference to the drawings in which:

FIGS. 1A and 1B show example images of corneal specular reflections in eye imaging before and after corneal flap removal of the eye of a patient;

FIG. 2 shows a block diagram for a surgical system to remove corneal specular reflections according to one example embodiment;

FIG. 3 is a flow diagram detailing a method of imaging an eye of a patient to produce eye images without corneal specular reflections according to one embodiment;

FIG. 4 shows an example of timing synchronization for pulsed infrared LED illumination and torsion camera imaging according to an example embodiment;

FIGS. 5A and 5B show example images captured in pulsed illumination with first and second light sources and resulting specular reflections;

FIGS. 6A and 6B show example images of FIGS. 5A and 5B after specular reflection removal processing according to various embodiments;

FIG. 7 shows an example embodiment of combined imaging from the processing of images of FIGS. 6A and 6B; and

FIG. 8 shows a method of eye imaging without corneal specular reflections for iris registration according to an embodiment.

### DETAILED DESCRIPTION OF THE PREFERRED EMBODIMENTS

Embodiments disclosed herein generally provide systems and methods for improving eye tracking and motion compensation during an ophthalmic diagnostic and/or treatment procedure. Laser-based systems are commonly used in ophthalmic procedures. Wavefront measurement systems may be utilized to measure the refractive characteristics of a patient's eye and to generate corrective procedure patterns for ablative photodecomposition, whereby a laser is used to ablate corneal tissues to correct vision defects. Measurement/diagnostic and treatment procedures may frequently be performed as different phases of a surgical procedure. For wavefront measurement and treatment to be imposed on the patient's eye, an accurate tracking system is required. For example, when wavefront measurement is taken, a patient may be in a seated position. Subsequently during a treatment phase, the patient may be in a supine (or laying down) position. Differences in patient position, and other factors, may contribute to differences in eye position, for example a different torsional orientation. Additionally, even when a patient does not change position between measurement and treatment phases, the eye may encounter differences in pupil center shift and/or cyclotorsional rotation, i.e., rotation of an eye around its visual axis. Differences between eye position during measurement and treatment may lead to vision correction inaccuracies.

In certain embodiments, eye tracking and motion compensation is used to stabilize the motion of the patient or organ (e.g., the patient's eye) between measurement and treatment phases. An automated target acquisition and tracking system allows a surgeon/user to predetermine a treatment pattern, for example, determining a laser firing pattern for ablation used in corneal reshaping. One tracking and motion compensation technique is referred to as iris registration (IR), where distinct features of a patient's iris may be used to provide common positional coordinate reference points between measurement and treatment phases. Iris registration (IR), and/or other types of tracking procedures, may capture images with a torsion camera to identify and/or match the reference points of a patient's eye for measurement and treatment phases.

Embodiments relate to new methods that use pulsed LED illumination and image processing algorithm to remove the corneal specular reflections in the laser eye images for iris registration. One embodiment uses a pulse train to alternatively turn on an auxiliary LED bank and a peripheral LED bank for torsion imaging. The torsion camera video frame acquisition may be synchronized with the pulsed LED illumination to capture two eye images sequentially, one eye image is illuminated only by the auxiliary LED bank and the following eye image is illuminated only by the peripheral LED bank. An image processing algorithm is used to remove the specular reflection in the two eye images and create a new eye image without specular reflection for iris registration. According to one embodiment, an excimer laser may use pulsed LED illumination along with software processing to remove corneal specular reflections in torsion images for iris registration.

Embodiments discussed herein may be utilized with any system where eye imaging and/or lasers are used in detection for diagnosis and/or surgical procedures on a patient's eye. Although not limited thereto, embodiments described herein may be adapted for use on a STAR S4IR® excimer laser platform. In such an implementation, the infrared LED illumination is changed from continuous illumination to pulsed LEDs illumination, and the current 30 Hz torsion camera imaging is upgraded to at 60 Hz or more for faster camera captures used for torsional eye imaging. Electric circuit updates may also be implemented to create the pulse signal to control the synchronization of the infrared LEDs illumination and the torsional camera for capturing images as detailed further below. Image processing software is implemented to remove the specular refractions from first and second images and create a new, or third, eye image for iris registration.

Embodiments may also be used on a femtosecond laser platform for eye registration during a cataract surgery or lenticule extraction surgery. Embodiments may also be used for eye imaging on a diagnostic device for eye examination if the 1st Purkinje image affects the accuracy of eye image detection. The imaging illumination wavelength may range from visible to infrared.

There is no limitation on processors used for eye registration. However, when embodiments are used for a real-time eye tracking or iris tracking, a reasonably fast processor may be needed for image processing in real-time. Processors and/or controllers, may be provided through the use of dedicated hardware as well as hardware capable of executing software in association with appropriate software. When provided by a processor, the functions may be provided by a single dedicated processor, by a single shared processor, or by a plurality of individual processors, some of which may be shared. Moreover, explicit use of the term “processor” or “controller” should not be construed to refer exclusively to hardware capable of executing software, and may implicitly include, without limitation, digital signal processor (DSP) hardware, network processor, application specific integrated circuit (ASIC), field programmable gate array (FPGA), read only memory (ROM) for storing software, random access memory (RAM), and nonvolatile storage. Other hardware, conventional and/or custom, may also be included.

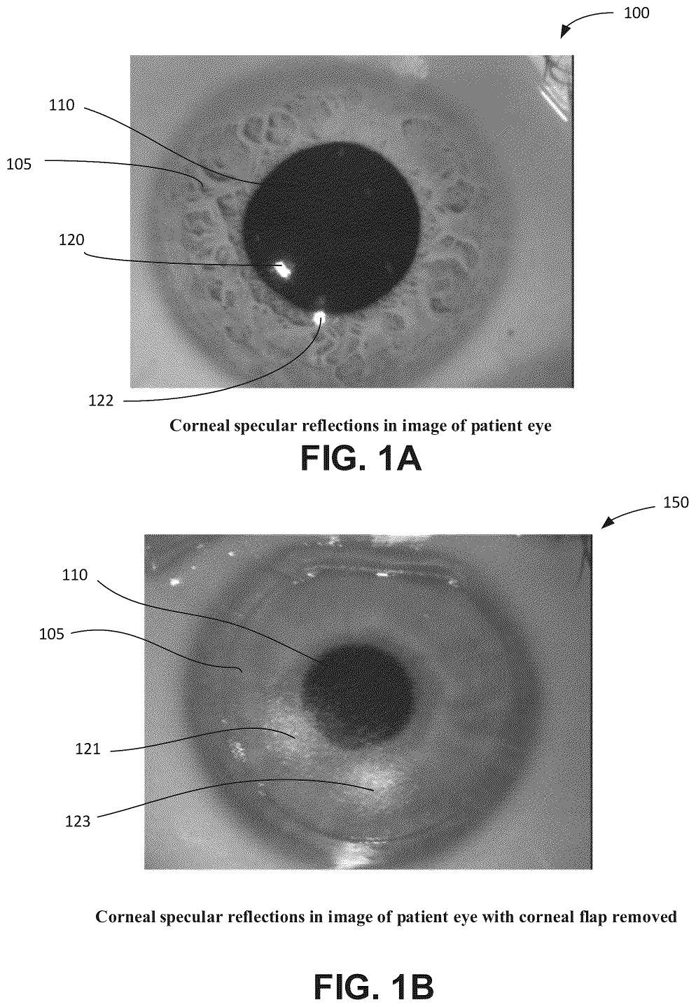

Referring to FIG. 1A, shows an example image 100 of an eye taken by a torsion camera. As one example, the center of pupil 110 may shift with respect to the surface of the cornea as the pupil size changes from scotopic to photopic lighting conditions. As previously mentioned, iris registration (IR) is used to compensate for the pupil center shift and cyclorotation of the eye between the wavefront exam and laser surgery. In certain systems, such as a STAR S4 IR® excimer laser system, two light sources, e.g., two infrared LED banks, may be used in imaging each eye: an auxiliary LED bank (AUX LED) and a peripheral LED (e.g., OD LED for the right eye illumination or OS LED for left eye illumination). Conventionally, these light sources are used to provide continuous illumination for an image capture device, such as a torsion camera, to capture a one frame laser image for iris registration. As can be seen in FIGS. 1A and 1B, eye images 100 and 150 captured by the torsion camera may show cornea specular reflections 120, 122 and 121, 123 from the light sources.

FIG. 1A shows specular reflections 120, 122 of the light sources formed on a smooth corneal surface in image 100 and FIG. 1B shows similar reflections 121, 123 in image 105 of the light sources formed on a rough stroma surface after flap lifted for corneal reshaping. In the example system capturing image 150 of FIG. 1B, the flap is created by a femtosecond laser system, although traditional scalpel or other techniques may be used to access the stroma for treatment. It should be noted that some systems may no longer even require flap removal for corneal reshaping, such as a PRK procedure or a corneal lenticule extraction procedure, and image 150 is shown merely for purposes of example showing how specular corneal reflections occur on a surgical eye when corneal flap is lifted. In any event, these specular reflections 120, 122 and 121, 123 can affect the accuracy of eye image detection and tracking by reducing the number of available match points of the eye, e.g., iris or pupil, for tracking and registration between imaging from the diagnostics device and imaging for laser treatment.

The treatment accuracy of laser surgery relies on highly precise eye image detection and precise iris registration. Various attempts have been made to address specular reflections including the polarized illumination of the previously cited published patent application US2090275929. However, prior solutions have drawbacks as noted previously.

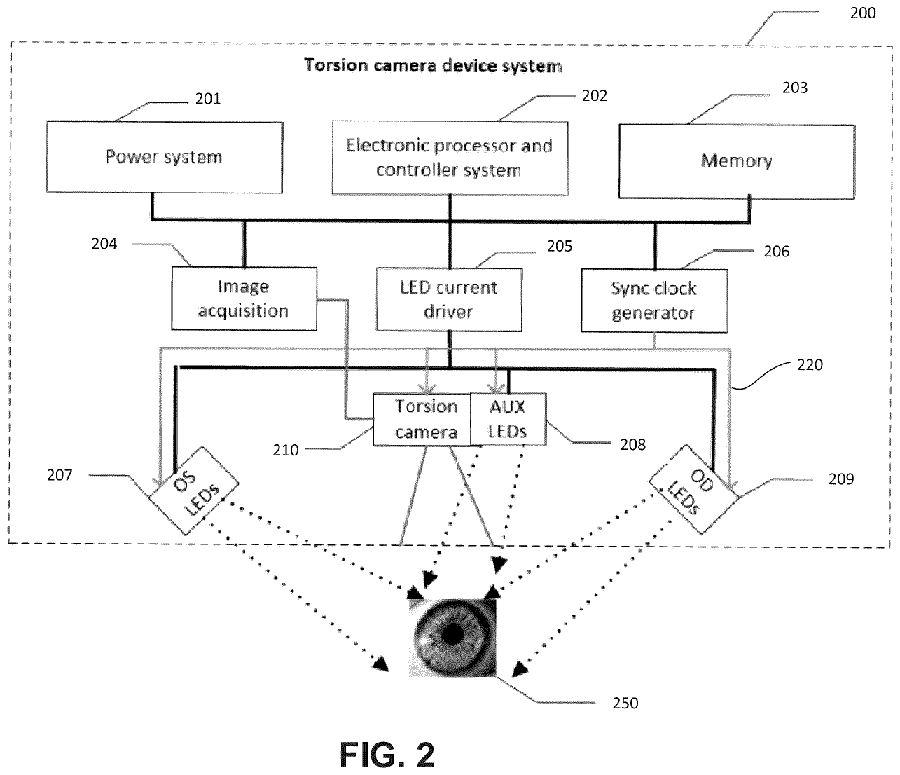

Referring to FIG. 2, a functional block diagram of a system 200 for imaging without corneal specular reflections is shown according to one example embodiment. In one example embodiment, system 200 may include a power system 201 for providing power to a control and processing system 202, memory 203, camera 210, sync clock trigger signals 220, image acquisition circuit 204, lighting current driver 205, clock generator 206 and a lighting system. The lighting system may include two or more light sources including, for example, an OS light 207, an auxiliary light 208, and OD light 209 triggered based on sync clock trigger signals 220. Synch clock trigger signals 220 may also trigger torsion camera 210 image acquisition. Lighting current driver 205 may be internal or external to the lighting system to provide power for illuminating the light sources of lighting system. In one embodiment, the lighting system may include OS light 207, auxiliary light 208 and OD light 209 as respective infrared LED banks, although other types of lighting are possible.

Synchronizing pulse (SYNCH PULSE) 220 may be generated by clock generator 206 to alternatingly activate and deactivate light sources of the lighting system, e.g., the auxiliary LED 208 and peripheral LED bank (e.g., OS LED 207 and/or OD LED 208), as well as activate torsion camera 210 for video frame acquisition during illumination and image capture of patient eye 250 according to the methods described herein.

As shown in FIG. 2, OS LED 207 (or OD LED 209) are physically separated from AUX LED 208 so as to produce corneal specular reflections at different locations in captured images of eye 250.

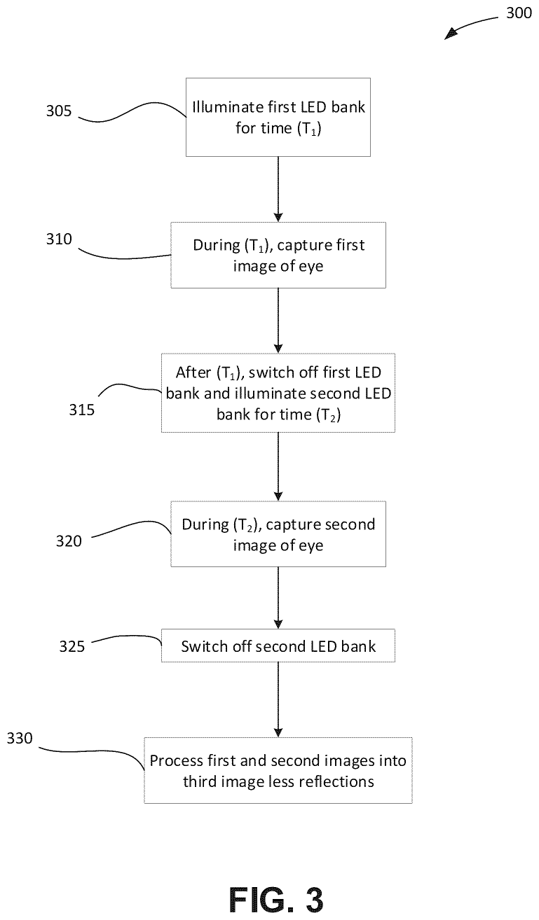

Turning to FIG. 3, an example method 300 of eye imaging according to one embodiment, includes illuminating 305 a first LED bank for time T₁ and during T₁, capturing 310 a first image of an eye. At step 315, the first LED bank is turned off and a second LED bank is illuminated for time T₂. During time T₂, the method includes capturing 320 a second image of the eye, and after T₂, the second LED bank is switched off 325. The timing range for image capture during T₁ and/or T₂ may be determined and balanced between the captured image pixel gray level and fuzziness of image pixels. The less of the exposure time, the minimum of moving image fuzziness, but the less image pixel gray value. While exposure timing may be preference specific, the sum of T₁ and T₂ is preferably within a 60 Hz pulsed cycle for considerations of potential eye movement as explained in greater detail below.

The first and second images are processed 330 with respective corneal light reflections from first and second LED banks removed. Processing 330 may generate a third image without light reflections present on the corneal area by combining processed first and second images. When the first and second LED banks are geographically displaced in the system of FIG. 2, the first image will have a light reflection from the first LED bank in the first image located at a different area (region of interest (ROI)) than a light reflection in the second image from the second LED bank, and the images can be combined by overlaying first and second images of the eye with reflection areas omitted from each of the first and second images. In this manner, a full area of an iris may be used for eye-tracking processing for subsequent diagnosis or surgical procedures to be performed. In one embodiment corresponding ROIs of first and second images having light reflections may simply be removed and when images are combined, the removed portions of images are supplemented by the corresponding unremoved ROI that is present in the other image when combined. In other embodiments, processing 330 may include transferring image data from one image to the other to supplement ROIs with removed data due to removal of specular reflections, and then first and second images are combined to create a third image with improved definition and lighting.

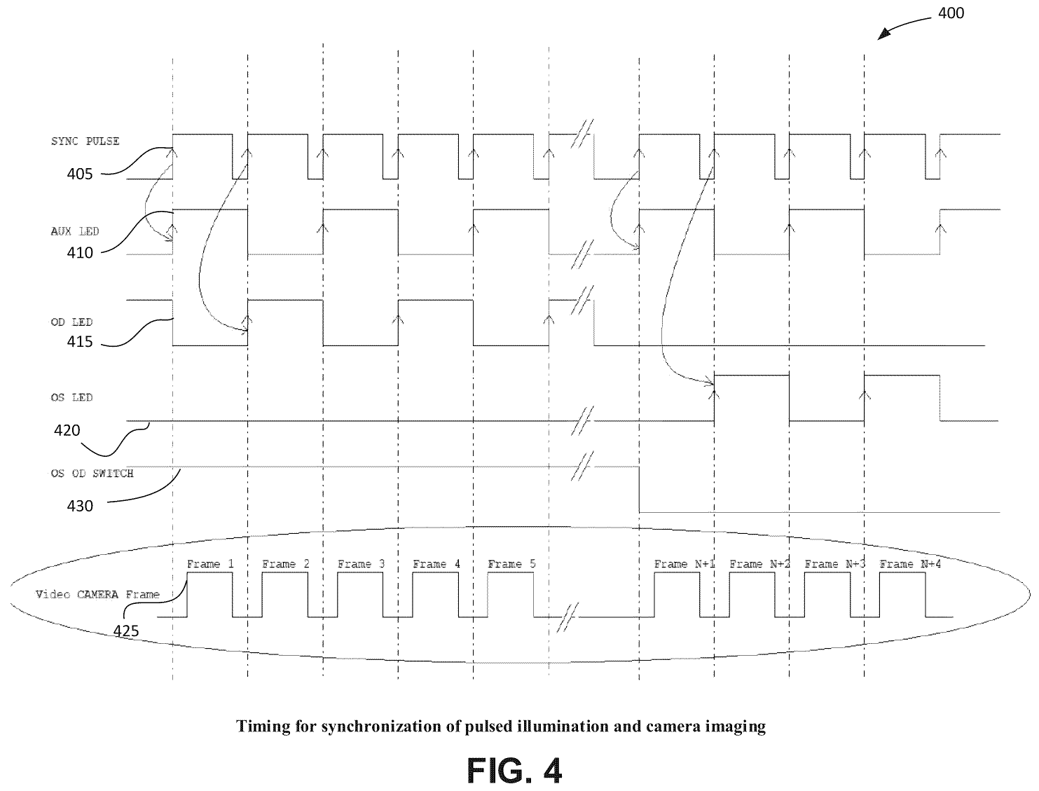

FIG. 4 illustrates an example timing diagram for the control of the pulsed LED illumination and the synchronized camera for image capturing. The timing of illumination and image capturing for the right eye is shown on the left part FIG. 4, while the timing of illumination and image capturing for the left eye is shown on the right part in FIG. 4. The right eye and left eye LED illumination may be controlled by an OS/OD switch signal. The example timing diagram 400 of FIG. 4 may be used for operation of the system 200 of FIG. 2 using, for example, method 300 of FIG. 3. In FIG. 4, a synchronization (SYNCH) pulse 405 may be utilized to trigger auxiliary LED signal 410, OD LED signal 415 and video capture frame signal 425.

For embodiments where both eyes of a patient will be imaged, the rising edge of the synchronizing pulse (SYNC PULSE) 405 is used as an external trigger for torsion camera to capture two eye images that one image is illuminated only by the auxiliary LED bank (AUX LED) and a subsequent image is illuminated only by the peripheral LED bank (OD LED or OS LED). To obtain more gray level matched video frame images in the field of view (FOV) of the camera, LED banks driving current may be adjusted or calibrated based on an obtained image histogram.

SYNCH pulse 405 may be used to trigger OS LED signal 420, and optionally, switch signal 430 to change use of OD to OS LEDs. For example, OD LED signal 415 is switched off and OS LED signal is switched on based on switch signal 430. A timer or alternative implementation for switching image lighting may also be used. As shown, video capture frame signal 425 may have an offset from respective aux LED and OD/OS LED illumination trigger signals to ensure an eye image has enough exposure level before image capture. The offset value of time delay is used to control the exposure time of one frame image based on image gray level (histogram value). The delay time of the camera used in practice, is 29 μs after the rising edge of the input trigger pulse. The offset between light activation and the image capture image may be calibrated in the system by tuning the LED light PWM control and LED current limiting resister. Calibration and tuning techniques are well known in the art and details are not discussed here.

Four frames may be used for image processing, one pair of images for OD and one pair of images for OS. Timing diagram 400 of FIG. 4 is for illustration purposes only and multi-frames of image could be used for image averaging to improve image quality providing no eye moving for these images. Timing diagram 400 illustrates that pulse signals may be continually sent to the LED driver to control the on/off of LEDs illumination, as well as to the torsion camera as external trigger signal to synchronize the LED illumination and camera image acquisition. The software will send the command to capture only two sequential images from each surgical eye (OD or OS).

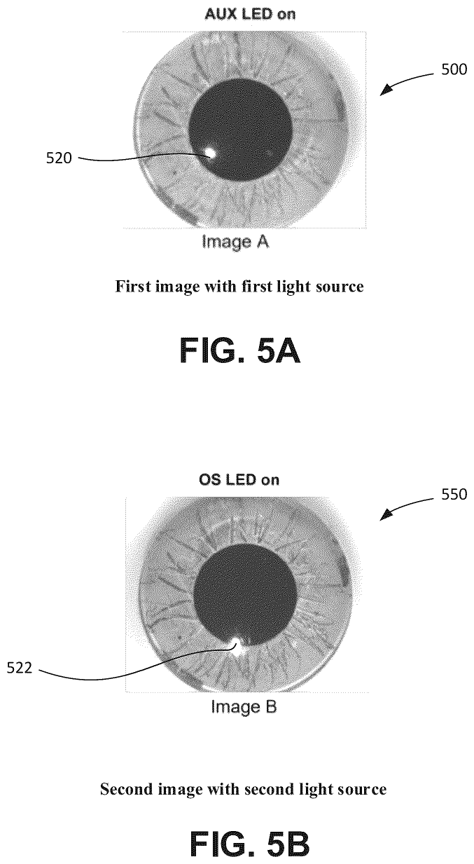

FIGS. 5A and 5B show illustrative examples of two torsion images 500 and 550 captured by the torsion camera from one test eye illuminated by the left eye's pulsed infrared LED illumination. A fast frame rate CMOS camera operating at a frame rate of greater than 60 Hz, and preferably ≥100 Hz, may be used for torsion imaging to avoid the eye motion in between the two eye images. First image 500 and second image 550 are captured with respective first and second light sources (e.g., image 500 with auxiliary LED on and image 550 with OS LED on). As can be seen, because of different physical positions of the auxiliary and OS light sources, different reflections 520 and 522 from the different light sources appear in the different eye image locations in first image 500 and second image 550.

Reflections 520 and 522 can then be removed or omitted in digital processing. An image processing algorithm may be used to remove the specular reflections 520, 522 resulting from the infrared LEDs in FIG. 5A and FIG. 5B.

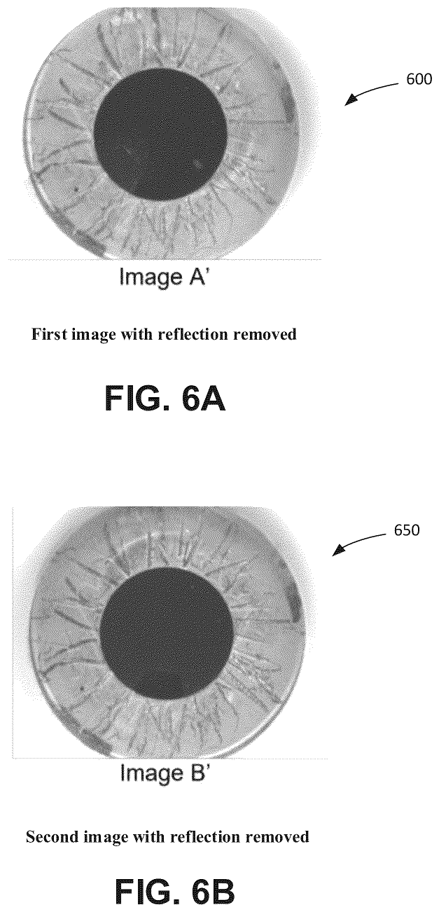

FIG. 6A shows image 600, which is a reflection removed processed image from image 500 of FIG. 5A. FIG. 6B shows image 650, which is a reflection removed processed image from image 550 of FIG. 5B. In one embodiment, the processed image 600 of FIG. 6A, a circular sector region of interest (ROI) in image 500 of FIG. 5A, with the specular reflections 520 from the auxiliary LED bank (AUX LED), is replaced by the same circular sector ROI of image 550 of FIG. 5B. In image 650 of FIG. 6B, the circular sector ROI in image 550 of FIG. 5B with the specular reflections 522 from the peripheral LED bank (OS LED in this case), is replaced by the same circular sector ROI of image 500 of FIG. 5A.

Because reflection removed processed images 600 and 650 may have iris detail obscured at points where reflections are removed, when images 600 and 650 are combined, full iris detail where reflections were once present, will be available for tracking in diagnosis and surgical procedures.

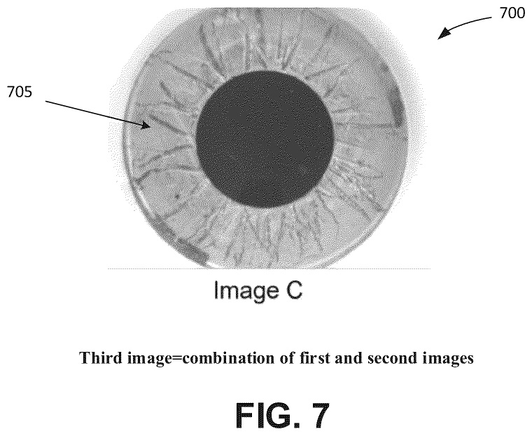

FIG. 7 shows an example image 700 resulting from the combination of images 600 and 650 from FIGS. 6A and 6B. Image 700 shows better image quality that compensates for any non-uniform illumination in image 600 of FIG. 6A only from the pulsed LED illumination with auxiliary LED bank (AUX LED), and any non-uniform illumination in image 650 of FIG. 6B only from the pulsed LED with the peripheral LED bank (OS LED in this case). Image 700 of FIG. 7 may thus have the comparable image quality for iris registration demonstrating full and uniformly lit intricate details of a patient's entire iris/pupil, even at points where reflections were once present. In this manner, more accurate eye tracking may be achieved without the costly and complicated disadvantages of prior solutions previously discussed.

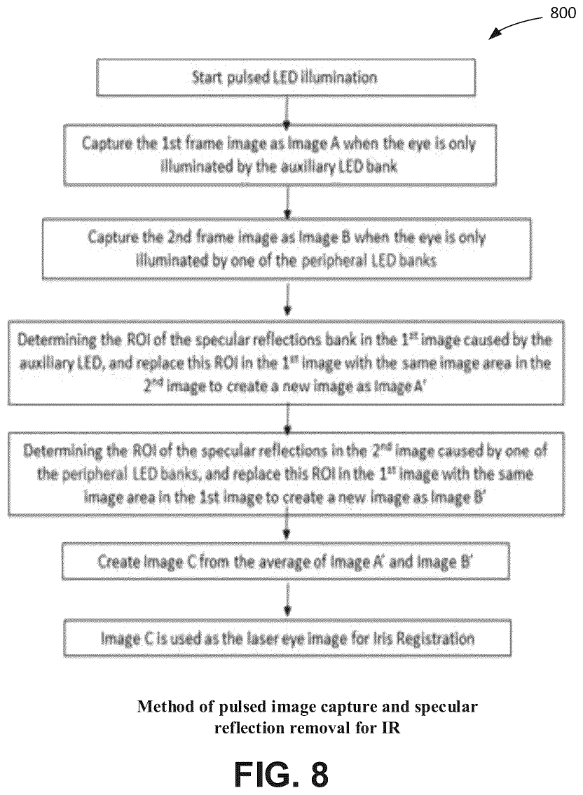

FIG. 8 shows a method 800 for the image processing that is used to remove the specular reflections in torsion imaging for iris registration and, in one example, can be implemented to the current production code (visual C++ based) for Iris Registration.

Although only one torsional image is captured on the laser system which is used for the iris registration, eye motion may occur during the camera exposure time may lead to anomolies in the capture eye image. To avoid the eye motion effect on the torsional eye imaging on the laser system, such as the STAR S4IR®, the frame rate for torsion eye imaging conventionally used for continues illumination is set at 30 Hz. Because embodiments described herein capture two sequential eye images to create a frame eye image for iris registration, the minimum frame rate should be increased to at least 60 Hz, which is twice faster than the current 30 Hz torsional camera. In some preferred embodiments, 100 Hz is used for the camera frame rate due to camera capability and preferred imaging results.

Embodiments disclosed herein may be in eye tracking apart from iris registration or used in a diagnostic devices apart from laser surgical systems unless the treatment plan is created by the diagnostic device and an eye image is associated for eye registration. In some embodiments, eye tracking tracks the pupil center movement. If the specular reflection falls inside the pupil area or at the pupil boundary, then the specular reflections can affect the pupil center detection.

Embodiments may alternatively be used in a method for eye imaging with a diagnostic device for eye examination, for example, if a first Purkinje image affects the accuracy of eye image detection.

Embodiments disclosed herein may be used for the iris registration in excimer laser systems as described above, or on a femtosecond laser system for cataract surgery or combination systems. In another case, embodiments disclosed herein can be used for the iris registration or lenticule centration on a femtosecond laser system for lenticule removal procedure. Further, embodiments may be used for eye tracker with pupil tracking and/or iris tracking.

Although features and elements are described above in particular combinations, one of ordinary skill in the art will appreciate that each feature or element can be used alone or in any combination with the other features and elements. In addition, the methods described herein may be implemented in a computer program, software, or firmware incorporated in a computer-readable medium for execution by a computer or processor. Examples of computer-readable media include electronic signals (transmitted over wired or wireless connections) and computer-readable storage media. Examples of computer-readable storage media include, but are not limited to, a read only memory (ROM), a random access memory (RAM), a register, cache memory, semiconductor memory devices, magnetic media such as internal hard disks and removable disks, magneto-optical media, and optical media such as CD-ROM disks, and digital versatile disks (DVDs).

What is claimed is:

1. A method of imaging for an eye for a surgical procedure, the method comprising:
capturing, using a torsion camera, a first image of the eye while the eye is illuminated with a first light source;
within a subsequent time period from the capturing of the first image, capturing a second image of the eye while the eye is illuminated with a second light source, different from the first light source;
processing the first and second images into a third image to remove specular reflections associated with the first light source and the second light source.

2. The method of claim 1, wherein the first light source is an auxiliary light emitting diode (LED) and the second light source is OD LED or OS LED for respective right eye or left eye imaging.

3. The method of claim 2, wherein the first and second light sources are separated to cause a displacement between a reflection in the first image and a reflection in the second image.

4. The method of claim 1, wherein a synchronization pulse triggers illumination of the first light source and capturing the first image.

5. The method of claim 4, wherein the synchronization pulse triggers illumination of the second light source and capturing the second image subsequent to triggering illumination of the first light source and capturing the first image.

6. The method of claim 5, wherein the synchronization pulse operates at a frequency of at least 60 Hz.

7. The method of claim 1, wherein the third image is used for an iris registration.

8. A surgical system for imaging an area of eye of a patient, the surgical system comprising:
an imaging light including a first light source and a second light source;
a synchronization circuit configured to receive or generate a pulsed signal to illuminate the first light source for a first time period and illuminate the second light source for a second time period, different than the first time period;
a torsion camera configured to capture a first image of the area during the first time period, the first image having a first light reflection from the first light source, and capture a second image of the area during the second time period, the second image having a second light reflection from the second light source; and
a processor configured to generate a third image based on the captured first image and second image, the third image having no specular light reflections.

9. The surgical system of claim 8, wherein the area comprises an iris.

10. The surgical system of claim 8, wherein the first light source and the second light sources are arranged in the imaging light to produce reflections captured in images at different locations of the area.

11. The surgical system of claim 10, wherein the pulsed signal has a cycle of greater than 60 Hz.

12. The surgical system of claim 8, wherein the imaging light source includes a third light source to capturing an image of an opposing eye of the patient, the third light source being triggered by the pulsed signal.

13. The surgical system of claim 8, wherein the first light source and the second light source comprise infrared light emitting diodes (LEDs).

14. The surgical system of claim 8, wherein the surgical system comprises one of an excimer or femtosecond laser surgical system.

15. The surgical system of claim 8, wherein the processor is configured to remove specular light reflections from the captured first and second images and combine first and second images less reflections into the third image.
